(19) United States
(12) Patent Application Publication
(10) Pub. No.: US 2002/0025310 A1
(43) Pub. Date: Feb. 28, 2002

(54) Compositions and methods for promoting healthy joints

(76) Inventor: Jeffrey S. Bland, Fox Island, WA (US)

Correspondence Address: GRANT R CLAYTON, CLAYTON HOWARTH & CANNON, PC, P O BOX 1909, SANDY, UT, 84091-1909, US

(21) Appl. No.: 09/776,579

(22) Filed: Feb. 2, 2001

Related U.S. Application Data
(60) Provisional application No. 60/179,885, filed on Feb. 2, 2000.

(51) Int. Cl. A61K 38/43; A61K 31/198; A61K 31/445; A61K 31/385; A61K 33/32

(52) U.S. Cl. 424/94.1; 424/643; 514/356; 514/436; 514/562

(57) ABSTRACT

A dietary supplement for promoting healthy joints and joint tissues is disclosed. The dietary supplement comprises a member selected from the group consisting of niacinamide, niacin, and mixtures thereof and an antioxidant nutrient. Illustrative antioxidant nutrients include N-acetylcysteine, lipoic acid, coenzyme Q10, vitamin E, carotenoids, and zinc. The dietary supplement supports optimal function and health of joints and joint tissues by interrupting a cascade of events involving reactive oxygen species, cytokines, and poly(ADP-ribose) synthetase, which self-amplifying, positive feed-forward cycle is inversely correlated with joint tissue integrity. A method for promoting healthy joints and joint tissues is also described.

### CROSS-REFERENCE TO RELATED APPLICATIONS

This application claims the benefit of U.S. Provisional Application No. 60/179,885, filed Feb. 2, 2000.

### STATEMENT REGARDING FEDERALLY SPONSORED RESEARCH OR DEVELOPMENT

Not applicable.

### BACKGROUND OF THE INVENTION

This invention relates to dietary supplements. More particularly, this invention relates to compositions and methods for providing nutritional support for healthy joints. In its most basic form, the invention comprises a mixture of niacinamide or niacin and an antioxidant nutrient, such as N-acetylcysteine, in a formula designed to support the optimal function and health of joints and joint tissues.

Increasing age has historically been known as a risk factor for osteoarthritis and joint “wear and tear,” its probable cause. Age-related changes in cartilage extracellular matrix may increase susceptibility to osteoarthritis, but supporting evidence is lacking. The progression of cartilage degeneration after severe joint damage from a trauma indicates that, from the point of damage onward, chondrocytes fail to uphold the integrity of the joint matrix. They begin to produce inappropriate non-cartilage-specific matrix constituents. In doing so, “joint space lubricants” like the proteoglycans are modified and cannot be replenished.

Recent studies indicate it is not trauma alone that can result in the damage to the production of the proteoglycans that help maintain joint dynamics. It is also their chemical modification by glycation. Glycation is the process by which a reducing sugar, such as glucose, is covalently linked to proteins, modifying their structure and functions. The non-enzymatic glycation of cartilage is known to increase in diabetes and other conditions associated with poor control of blood sugar. Studies now indicate that increased levels of ribosylated cartilage are associated with reduced proteoglycan synthesis and increased risk for degenerative joint disease. J. DeGroot et al., Age-related Decrease in Proteoglycan Synthesis of Human Articular Chondrocytes, 42 Arthritis Rheum. 1003-1009 (1999).

Because increasing age is associated with increasing glycation of proteins, it is suggested that one of the variables linking age with degenerative joint disease is the glycation of cartilage that reduces the production of proteoglycans involved in joint lubricants. Proteoglycans are made up in part of amino sugars, such as glucosamine. The salvage synthesis of proteoglycans may therefore depend on increased availability of amino sugars like glucosamine for repletion. This may be particularly true in cases where increased cartilage glycosylation occurs in older individuals, and it may explain clinical reports of the effectiveness of glucosamine administration to manage manifestations of osteoarthritis and degenerative joint problems. C. L. Deal et al., Nutraceuticals as Therapeutic Agents in Osteoarthritis: The Role of Glucosamine, Chondroitin Sulfate, and Collagen Hydrolysate, 25 Rheum. Dis. Clin. North Am. 379-395 (1999).

As the pain associated with osteoarthritis develops, individuals frequently assume a more sedentary lifestyle. In the past, immobilization of the affected joint and “resting” it to allow it to recover was even a suggested therapy. It is now known that exercise may play an important role in maintaining cartilage integrity. I. Kiulranta et al., Moderate Running Exercise Augments Glycosaminoglycans and Thickness of Articular Cartilage in the Knee Joint of Young Beagle Dogs, 6 J. Orthop. Res. 188-195 (1988). Immobilizing the joint leads to loss of proteoglycans and results in what is called “disuse atrophy.”

Joint motion alone is not enough to maintain the health of the articular cartilage. In dogs with free movement in the knee, but with the knee unloaded, proteoglycan is depleted in the knee cartilage. This demonstrates that motion in the absence of loading fails to maintain proteoglycan. At the other extreme, excessive loading can also compromise the cartilage. Dogs running 40 kilometers daily on a slight incline for one year showed local softening of the articular cartilage and decreased proteoglycan. With moderate running, however, proteoglycan content and cartilage thickness were enhanced in these dog studies. I. Kiulranta et al., supra.

Between immobilization and excessive joint use is a broad range of activity levels that maintain normal cartilage. Animal studies revealed that a sedentary lifestyle led to a lower proteoglycan content in the cartilage and lower synovial fluid volume. These changes were associated with cartilage fibrillation, pitting, and fissuring. Daily exercise at a moderate level prevented early cartilage deterioration and maintained normal articular cartilage and proteoglycan synthesis.

The traditional pharmacological approach to management of osteoarthritis discomfort is the chronic administration of nonsteroidal antiinflammatory drugs (NSAIDs) to uncouple the inflammatory cycle by blocking cyclooxygenase and preventing the formation of downstream inflammatory mediators. NSAIDs are among the most widely used drug; more than 70 million prescription and more than 30 billion over-the-counter NSAIDs are sold annually in the United States alone.

Although NSAIDs are generally well tolerated, adverse gastrointestinal events in a small but significant percentage of patients result in substantial morbidity and mortality. Evidence now indicates that at least 10 to 20 percent of patients taking NSAIDs have dyspepsia. The rate of NSAID-related serious gastrointestinal complications requiring hospitalization has decreased in recent years. This decrease may be due, in part, to medical education campaigns that have persuaded physicians to use newer, less toxic NSAIDs in high-risk populations. The mortality rate among patients hospitalized for NSAID-induced upper GI bleeding is about 5 to 10 percent. In the United States, it is estimated that NSAIDs are used regularly by at least 13 million people with various types of inflammatory arthritis.

Based on these data, the annual number of hospitalizations in the United States for serious GI complications is estimated to be at least 103,000. At an estimated cost of $15,000 to $20,000 per hospitalization, the annual direct costs of such complications exceed $2 billion. A conservative estimate is that 16,500 NSAID-related deaths occur among patients with rheumatoid arthritis or osteoarthritis every year in the United States.

An added concern, beyond the risk of gastropathy with the use of NSAIDs, is nephropathy. A recent report indicates that for older people who use NSAIDs for an extended period of time the risk of renal damage may be as great or greater than the risk of gastropathy. T. S. Field et al., The Renal Effects of Nonsteroidal Antiinflammatory Drugs in Older People: Findings from the Established Population for Epidemiological Studies of the Elderly, 47 J. Am. Geriatr. Soc. 507-511 (1999).

Medications with antiinflammatory or aspirin-like activity but without the same risk of gastropathy have recently been developed. Called COX-2 selective inhibitors, they operate by more selective inhibition of the COX-2 isoform rather than the COX-1 isoform of cyclooxygenase. Some concern has been expressed for individuals who have shifted their allegiance to “super aspirins” represented by the selective COX-2-inhibiting drugs. First is the suggestion that long-term administration of COX-2 selective inhibitors may still lead to renal problems. A second concern is that excessive suppression of COX-2 might have an adverse impact on the resolution phase of inflammation by inhibiting the synthesis of cyclopentenones that bind to peroxisome proliferator-activated receptor gamma (PPARγ) and effectively downregulate the inflammatory process. D. W. Gilroy et al., Inducible Cyclooxygenase May Have Anti-inflammatory Properties, 5 Nature Med. 698-701 (1999). Specific COX-2 inhibitors like CELEBREX (Searle, Monsanto) and VIOXX (Merck) may have adverse effects on normal physiological processes associated with pain if they are taken over a long period of time. K. Seibert et al., COX-2 Inhibitors-Is There Cause for Concern?, 5 Nature Med. 621-622 (1999).

Functional and nutritional medicine approaches for the modulation of inflammatory mediators, however, may reduce the need for medication and improve patient prognosis without the side effects associated with NSAIDs and COX-2 inhibitors.

In view of the foregoing, it will be appreciated that providing dietary supplements and methods of use thereof for promoting healthy joints and joint tissues while avoiding the side effects associated with NSAIDs and COX-2 inhibitors would be a significant advancement in the art.

### BRIEF SUMMARY OF THE INVENTION

It is an object of the present invention to provide dietary supplements and methods of use thereof for promoting healthy joints and joint tissues.

It is also an object of the invention to provide dietary supplements for inhibiting poly(ADP-ribose) synthetase, which is known to be upregulated in the inflammation process.

It is another object of the invention to provide dietary supplements and methods of use thereof for inhibiting poly(ADP-ribose) synthetase and thus counteracting the depletion of ATP that occurs in the inflammation process.

It is still another object of the invention to provide dietary supplements and methods of use thereof that inhibit the deleterious effects of oxygen free radicals, including activation of poly(ADP-ribose) synthetase.

These and other objects can be addressed by providing a dietary supplement for promoting health of joints and joint tissues comprising a mixture of an effective amount of a member selected from the group consisting of niacinamide, niacin, and the like, and mixtures thereof, and an effective amount of an antioxidant nutrient. The antioxidant nutrient is preferably a member selected from the group consisting of N-acetylcysteine, alpha lipoic acid, coenzyme Q10, vitamin E, carotenoids, zinc, and the like, and mixtures thereof. The dietary supplement can also further comprise a member selected from the group consisting of diluents, binders, lubricants, disintegrants, coloring agents, flavoring agents, and the like, and mixtures thereof.

A preferred formulation of the dietary supplement comprises about 10-90% by weight of the member selected from the group consisting of niacinamide, niacin, and mixtures thereof and about 10-90% by weight of the antioxidant nutrient. More preferably, the dietary supplement comprises about 30-85% by weight of the member selected from the group consisting of niacinamide, niacin, and mixtures thereof and about 15-70% by weight of the antioxidant nutrient. Most preferably, the dietary supplement comprises about 50-80% by weight of the member selected from the group consisting of niacinamide, niacin, and mixtures thereof and about 20-50% by weight of the antioxidant nutrient.

Another preferred embodiment of the invention comprises a method for promoting healthy joints and joint tissues comprising orally administering an effect amount of the dietary supplement. The effective amount of the dietary supplement comprises about 5-75 mg/kg/day.

### BRIEF DESCRIPTION OF THE SEVERAL VIEWS OF THE DRAWINGS

FIG. 1 shows a diagram of the cycle of events inversely associated with joint tissue integrity.

### DETAILED DESCRIPTION

Before the present compositions and methods for promoting healthy joints are disclosed and described, it is to be understood that this invention is not limited to the particular configurations, process steps, and materials disclosed herein as such configurations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

The publications and other reference materials referred to herein to describe the background of the invention and to provide additional detail regarding its practice are hereby incorporated by reference. The references discussed herein are provided solely for their disclosure prior to the filing date of the present application. Nothing herein is to be construed as an admission that the inventors are not entitled to antedate such disclosure by virtue of prior invention.

It must be noted that, as used in this specification and the appended claims, the singular forms “a,” “an,” and “the” include plural referents unless the context clearly dictates otherwise. Thus, for example, reference to a mixture containing “an antioxidant nutrient” includes reference to a mixture of two or more of such antioxidant nutrients, reference to “a dose” includes reference to one or more of such doses, and reference to “a lubricant” includes reference to two or more of such lubricants.

In describing and claiming the present invention, the following terminology will be used in accordance with the definitions set out below.

As used herein, “comprising,” “including,” “containing,” “characterized by,” and grammatical equivalents thereof are inclusive or open-ended terms that do not exclude additional, unrecited elements or method steps. “Comprising” is to be interpreted as including the more restrictive terms “consisting of” and “consisting essentially of.”

As used herein, “consisting of” and grammatical equivalents thereof exclude any element, step, or ingredient not specified in the claim.

As used herein, “consisting essentially of” and grammatical equivalents thereof limit the scope of a claim to the specified materials or steps and those that do not materially affect the basic and novel characteristic or characteristics of the claimed invention.

As used herein, “effective amount” means an amount of a dietary supplement that is nontoxic but sufficient to provide the desired effect and performance at a reasonable benefit/risk ratio attending any such dietary supplement. An effective amount of an antioxidant nutrient is an amount sufficient to inhibit or reduce the oxidative effects of oxygen free radicals.

As used herein, “antioxidant nutrient” means a nutrient that is safe and effective for human consumption and that exhibits activity as an antioxidant. Illustrative antioxidant nutrients include N-acetylcysteine, alpha lipoic acid, coenzyme Q10, vitamin E, carotenoids, zinc, and the like, and mixtures thereof.

As used herein, “tablets” are solid pharmaceutical dosage forms containing nutrient substances with or without suitable diluents and prepared either by compression or molding methods well known in the art. Tablets have been in widespread use since the latter part of the 19^{th} century and their popularity continues. Tablets remain popular as a dosage form because of the advantages afforded both to the manufacturer (e.g., simplicity and economy of preparation, stability, and convenience in packaging, shipping, and dispensing) and the patient (e.g., accuracy of dosage, compactness, portability, blandness of taste, and ease of administration). Although tablets are most frequently discoid in shape, they may also be round, oval, oblong, cylindrical, or triangular. They may differ greatly in size and weight depending on the amounts of nutrient substances present and the intended method of administration. They are divided into two general classes, (1) compressed tablets, and (2) molded tablets or tablet triturates. In addition to the active or therapeutic ingredient or ingredients, tablets contain a number or inert materials or additives. A first group of such additives includes those materials that help to impart satisfactory compression characteristics to the formulation, including diluents, binders, and lubricants. A second group of such additives helps to give additional desirable physical characteristics to the finished tablet, such as disintegrators, colors, flavors, and sweetening agents.

As used herein, “diluents” are inert substances added to increase the bulk of the formulation to make the tablet a practical size for compression. Commonly used diluents include calcium phosphate, calcium sulfate, lactose, kaolin, mannitol, sodium chloride, dry starch, powdered sugar, silica, and the like.

As used herein, “binders” are agents used to impart cohesive qualities to the powdered material. Binders, or “granulators” as they are sometimes known, impart a cohesiveness to the tablet formulation, which insures the tablet remaining intact after compression, as well as improving the free-flowing qualities by the formulation of granules of desired hardness and size. Materials commonly used as binders include starch; gelatin; sugars, such as sucrose, glucose, dextrose, molasses, and lactose; natural and synthetic gums, such as acacia, sodium alginate, extract of Irish moss, panwar gum, ghatti gum, mucilage of isapol husks, carboxymethylcellulose, methylcellulose, polyvinylpyrrolidone, Veegum, microcrystalline cellulose, microcrystalline dextrose, amylose, and larch arabogalactan, and the like.

As used herein, “lubricants” are materials that perform a number of functions in tablet manufacture, such as improving the rate of flow of the tablet granulation, preventing adhesion of the tablet material to the surface of the dies and punches, reducing interparticle friction, and facilitating the ejection of the tablets from the die cavity. Commonly used lubricants include talc, magnesium stearate, calcium stearate, stearic acid, and hydrogenated vegetable oils. Preferred amounts of lubricants range from about 0.1% by weight to about 5% by weight.

As used herein, “disintegrators” or “disintegrants” are substances that facilitate the breakup or disintegration of tablets after administration. Materials serving as disintegrants have been chemically classified as starches, clays, celluloses, algins, or gums. Other disintegrators include Veegum HV, methylcellulose, agar, bentonite, cellulose and wood products, natural sponge, cation-exchange resins, alginic acid, guar gum, citrus pulp, cross-linked polyvinylpyrrolidone, carboxymethylcellulose, and the like.

As used herein, “coloring agents” are agents that give tablets a more pleasing appearance, and in addition help the manufacturer to control the product during its preparation and help the user to identify the product. Any of the approved certified water-soluble FD&C dyes, mixtures thereof, or their corresponding lakes may be used to color tablets. A color lake is the combination by adsorption of a water-soluble dye to a hydrous oxide of a heavy metal, resulting in an insoluble form of the dye.

As used herein, “flavoring agents” vary considerably in their chemical structure, ranging from simple esters, alcohols, and aldehydes to carbohydrates and complex volatile oils. Synthetic flavors of almost any desired type are now available.

In a basic embodiment of the present invention, the dietary supplement comprises a mixture of niacinamide or niacin and an antioxidant nutrient.

Niacin (nicotinic acid) and niacinamide (nicotinamide) have identical properties as vitamins. In the body niacin is converted to niacinamide, which is an essential constituent of coenzymes I and II that occur in a wide variety of enzyme systems involved in anaerobic oxidation of carbohydrates. The coenzyme serves as a hydrogen acceptor in the oxidation of the substrate. These enzymes are present in all living cells and take part in many reactions of biological oxidation. Nicotinamide-adenine dinucleotide (NAD) and nicotinamide-adenine dinucleotide phosphate (NADP) are coenzymes synthesized in the body that take part in the metabolism of all living cells. Since they are of such widespread and vital importance, it is not difficult to see why serious disturbance of metabolic processes occurs when the supply of niacinamide or niacin to the cell is interrupted. Niacinamide and niacin are readily absorbed from the intestinal tract, and large doses may be given orally or parenterally with equal effect. Further, niacinamide and niacin improve circulation and reduce the cholesterol level in the blood; maintain the nervous system; help metabolize protein, sugar & fat; reduce high blood pressure; increase energy through proper utilization of food; prevent pellagra; and help maintain a healthy skin, tongue, and digestive system.

The mechanism by which niacinamide and niacin function in promoting joint health is by inhibiting the enzyme poly(ADP-ribose) polymerase (PARP). PARP is also known as poly(ADP-ribose) synthetase (PARS). Upregulation of the activity of PARP or PARS is well known to occur during the process of inflammation.

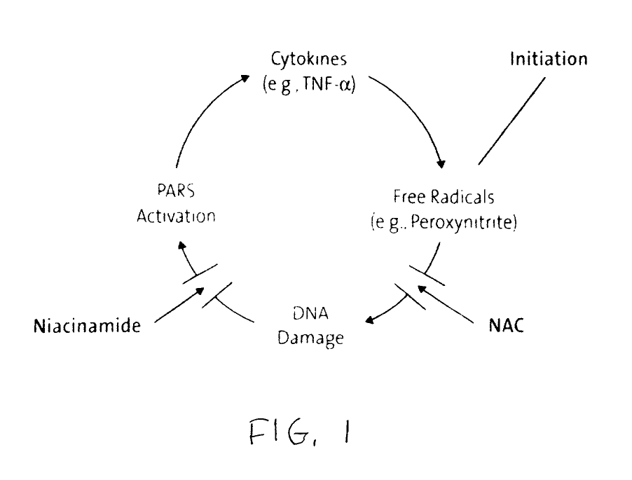

PARS is a protein-modifying nucleotide polymerizing enzyme. The trigger for PARS activation is a DNA strand break (FIG. 1), which can be induced by a variety of environmental stimuli and free radicals/oxidants, most notably, the hydroxyl radical and peroxynitrite. C. Szabo, Role of Poly(ADP-ribose) Synthetase in Inflammation, 350 Eur. J. Pharmacol. 1-19 (1998). Peroxynitrite is formed through the diffusion-controlled reaction of superoxide anion with nitric oxide associated with immune upregulation and oxidative stress. When PARS becomes activated, it uses NAD⁺ as a substrate and catalyzes the building of homopolymers of ADP-ribose. As a consequence, activation of PARS during inflammation initially depletes NAD⁺, which ultimately leads to a reduction of ATP. Clinically, this is often seen as the fatigue that occurs during viral infection or inflammation. Much cellular energy is being used in driving the PARS pathway. Inflammatory stimuli, which manifest themselves through the release of mediators from stimulated white cells, subsequently increase the production of peroxynitrite and hydroxyl radical. A result can be single-stranded DNA breaks and the upregulation of PARS, which, in turn, depletes ATP, resulting in cell death. As shown in FIG. 1, PARS activation is part of a cascade of events involving reactive oxygen species (ROS), cytokines, and damage to DNA. This cascade is a self-amplifying, positive feed-forward cycle that is inversely correlated with joint tissue integrity.

All animal models of inflammatory disorders indicate activation of the PARS pathway. Conditions like arthritis, inflammatory bowel disease, pancreatic islet cell destruction in diabetes, allergic encephalomyelitis/multiple sclerosis, Parkinson's disease, and vascular alterations in shock are associated with the inflammatory upregulation of PARS and depletion of ATP. The relationship between proinflammatory cytokines, the release of nitric oxide and superoxide, mitochondrial uncoupling, DNA strand breaks, and PARS activation with ATP depletion is described in C. Szabo, supra.

Niacinamide and niacin are precursors to NAD+and inhibitors of PARS, sparing cellular ATP in times of immune upregulation. Niacinamide has been demonstrated to be an NAD⁺ precursor in human erythrocytes and presumably also other types of cells. High concentrations of niacinamide can drive equilibrium toward the synthesis of NAD⁺, which minimizes the effect of PARS activation.

Doses of niacinamide required to achieve this inhibition may be in the range of several thousand milligrams per day. Recently, however, a clinical study indicated that by using niacinamide along with other nutrients to help facilitate control of hydroxyl radicals and peroxynitrite, lower levels of niacinamide might produce similar benefit. In this study, several hundred milligrams of niacinamide were administered daily along with zinc (30 mg/day), and mixed carotenoids (100 mg/day). Y. Sheng et al., DNA Repair Enhancement by a Combined Supplement of Carotenoids, Nicotinamide and Zinc, 22 Cancer Detect. Prev. 284-292 (1998).

Another study found that 100 mg/day supplementation with nicotinic acid (niacin) as a precursor to NAD⁺ did not result in a significant reduction of PARS activity in smokers who exhibited varying levels of cytogenic damage. These results suggest that the therapeutic dose for niacin/niacinamide is probably greater than 100 mg, but may be less than several thousand milligrams per day, depending on the overall balance of synergistic nutrients in which it is administered. G. J. Hageman et al., Nicotinic Acid Supplementation: Effects on Niacin Status, Cytogenetic Damage, and Poly(ADP-ribosylation) in Lymphocytes of Smokers, 32 Nutr. Cancer 113-120 (1998).

N-acetylcysteine (NAC) has been found to have a profound effect on T cell function in human subjects. The effects appear to be beyond the role of NAC as a precursor of glutathione and may relate to its ability to deliver active thiols that help modulate the intercellular mitochondrial redox system. Control of mitochondrial redox in immune cells, in turn, may influence its inflammatory and oxidant response to stimulus. NAC is also known to reduce the viscosity of pulmonary secretions and facilitate their removal. Hence, NAC has been used as adjuvant therapy in bronchopulmonary disorders when mucolysis is desirable. It is thought that the sulfhydryl group in NAC reduces the disulfide bonds in mucus and thus lowers the viscosity. The commonly used dose of NAC is 200 to 400 mg/day. This dose resulted in significant increase in intercellular glutathione levels and a reduction in the glutathione disulfide-to-reduced glutathione ratio.

NAC supplementation also helps to protect against age-related decline of oxidative phosphorylation and liver mitochondria. In an animal study, NAC-supplemented senescent mice had significantly higher specific activities of complex I, IV, and V in hepatic mitochondria compared to controls that did not receive NAC supplementation. J. Miquel et al., N-acetylcysteine Protects Against Age-related Decline of Oxidative Phosphorylation in Liver Mitochondria, 292 Eur. J. Pharmacol. 333-335 (1995). Thus, chronic dietary administration of N-acetylcysteine may contribute to the preservation of mitochondrial oxidative phosphorylation proteins in the liver of senescent mice because of their free radical scavenging action. Moreover, because of the ease of administration and low toxicity of N-acetylcysteine in humans, its potential usefulness for preservation of liver optimum bioenergetic competence and function in aging humans deserves investigation.

Another study evaluated the role of NAC supplementation in the management of chronic obstructive pulmonary disease (COPD), a condition known to be associated with increased oxidative stress. C. L. Van Herwaarden et al., The Role of N-acetylcysteine in the Treatment of Chronic Obstructive Pulmonary Disease, 47 Netherlands J. Med. 45-48 (1995). COPD patients who received 600 mg per day of oral NAC had fewer bacterial-positive cultures, reduced use of bronchodilators, and improved clinical course compared to patients who did not receive NAC supplementation. The investigators concluded that NAC therapy maybe very helpful for those conditions associated with higher levels of oxidative stress and the production of nitric oxide, peroxynitrite, and hydroxyl radical.

A study of individuals engaged in heavy aerobic exercise found that those who took 800 mg of NAC orally had less exercise-associated damage in leukocyte DNA, a lowered plasma lipid peroxide level, and increased glutathione-to-glutathione disulfide ratio. C. K. Sen et al., Oxidative Stress after Human Exercise: Effect of N-acetylcysteine Supplementation, 76 J. Applied Physiol. 2570-2577 (1994). The investigators suggested that the oxidative stress of heavy exercise may be reduced by NAC supplementation through the improved “redox buffering” provided by NAC.

The oral availability of NAC is known to vary between 6 and 10 percent; slow release tablets have the lowest and fast-dissolving tablets the highest bioavailability. NAC is a conditionally essential nutrient with a fairly broad dose/response safety margin. NAC is metabolized during its first pass in the gut wall and liver, and oral NAC offers prompt availability of thiol groups needed for glutathione synthesis in hepatic cells.

A study of NAC toxicity was conducted to evaluate doses of NAC at 400, 800, 1600, and 3200 mg/m²/day in divided doses, doubled at the end of each month, to a final dose of 6400 mg/m²/day. L. Borgstrom et al., Pharnacokinetics of N-acetylcysteine in Man, 31 Eur. J. Clin. Pharmacol. 217-222 (1986). The only significant side effects were bad taste and GI disturbances, and those were dependent on the individual. Only 13 of 26 individuals reported side effects, none of whom reported difficulty until a dose of 800 mg/m² was exceeded.

Animal studies demonstrate that NAC helps protect isolated alveolar cells against intracellular ATP depletion after oxidant exposure. Once again, this effect may be a consequence of protection of intracellular mitochondrial redox and downregulation of PARS activity.

Alpha-lipoic acid (technically known as DL-alpha lipoic acid) is a powerful antioxidant being researched for unique properties that may provide both preventive and therapeutic benefits in numerous conditions and diseases including diabetes, heart disease, and even possibly HIV infection. Lipoic acid and its reduced form, DHLA, show the ability to directly quench a variety of reactive oxygen species, inhibit reactive oxygen generators, and spare and regenerate other antioxidants. Lipoic acid not only protects the nervous system, but is also involved in regenerating nerves. It is also being studied in the treatment of Parkinson's disease and Alzheimer's disease. Lipoic acid is best known for its ability to help regenerate damaged liver tissue when nothing else will. Lipoic acid is marketed in Germany for treating diabetic neuropathy. It also has an essential role in mitochondrial dehydrogenase reactions. Alpha lipoid acid is preferably present in the formulation in the range of about 5-500 mg and, more preferably in the range of about 5-250 mg.

Coenzyme Q10 is an essential electron and proton carrier that fuctions in the production of biochemical energy in aerobic organisms. Coenzyme Q10 is found in every cell in the body, thus its other name, ubiquinone (from the word ubiquitous and the coenzyme quinone). The structure of coenzyme Q10 consists of a quinone ring attached to an isoprene side chain. Because the body must have energy available to perform even the simplest operation, coenzyme Q10 is considered essential for the body's cells, tissues, and organs. Coenzyme Q10 also has antioxidant and membrane stabilizing properties that serve to prevent the cellular damage that results from normal metabolic processes. Even though the body has the ability to produce coenzyme Q10, deficiencies have been reported in a range of clinical conditions. Aging is considered one reason for a deficiency, since the liver loses its ability to synthesize coenzyme Q10 as one gets older. Besides aging, poor eating habits, stress, and infection affect the body's ability to provide adequate amounts of coenzyme Q10. In addition to its function as an antioxidant and its salutary effect on joints, other known results of using coenzyme Q10 as an oral supplement are energy increase, improvement of heart function, prevention and cure of gum disease, a boost to the immune system, and possible life extension. AIDS is a primary target for research on coenzyme Q10 because of its immense benefits to the immune system. Further, coenzyme Q10 has also been reported to provide a beneficial effect in the treatment of breast cancer. Coenzyme Q10 is preferably present in the formulation in the range of about 1-100 mg and, more preferably, in the range of about 1-50 mg.

Vitamin E is a group of compounds (tocol and tocotrienol derivatives) that exhibit qualitatively the biological activity of α-tocopherol. Biological activity associated with the vitamin nature of the group is exhibited by four major compounds: α-, β-, γ-, and δ- tocopherol, each of which can exist in various stereoisomeric forms. The tocopherols act as antioxidants, δ-tocopherol having the greatest antioxidant power. The most critical function of vitamin E occurs in the membranous parts of the cells. Vitamin E interdigitates with phospholipids, cholesterol, and triglycerides, the three main structural elements of the membranes. Since vitamin E is an antioxidant, a favored reaction is with the very reactive and highly destructive free radicals. Free radicals are products of oxidative deterioration of such substances as polyunsaturated fat. Vitamin E converts the free radical into a less reactive and nonharmful form. Vitamin E also supplies oxygen to the blood, which is then carried to the heart and other organs; thus alleviating fatigue; aids in bringing nourishment to cells; strengthens the capillary walls and prevents the red blood cells from destructive poisons; prevents and dissolves blood clots; and has also been used in helping prevent sterility, muscular dystrophy, calcium deposits in blood walls, and heart conditions. Vitamin E is preferably present in the formulation in an amount in the range of about 200-800 international units (IU) and, more preferably, in the range of about 250-700 IU.

Carotenoids are a family of hundreds of plant pigments found in fruits and vegetables that are red, orange, and deep yellow in color, and also in some dark green leafy vegetables. See USDA-NCC Carotenoid Database for U.S. Foods (1998). Carotenoids are the precursors of most of the vitamin A found in animals. At least 10 different carotenoids exhibit provitamin A activity, including α- and β-carotenes and cryptoxanthin. As precursors of vitamin A, carotenoids exhibit an effect on vision, but carotenoids are known to have other beneficial effects in the diet, as well. For example, carotenoids are also known for their antioxidant activity in helping protect the body from free radical damage.

Volumes of research reveal that two carotenoids—lutein and zeaxanthin—are found in great concentrations in the macula of the eye. This research also indicates that maintaining high levels of these two carotenoids, especially lutein, may help diminish the effects of age-related macular degeneration, the leading cause of blindness in those over 65 years of age. Lutein acts as an antioxidant, protecting cells against the damaging effects of free radicals. As with the other carotenoids, lutein is not made in the body and, therefore, must be obtained from food or dietary supplements.

At one time researchers believed all antioxidants served the same purpose. Now there is growing evidence that individual antioxidants may be used by the body for specific purposes. Researchers believe that lutein is deposited into areas of the body most prone to free radical damage. One major example is the macula, a tiny portion of the retina. Research indicates that because of its antioxidant properties, lutein consumption may play a role in maintaining the health of the eyes, heart and skin as well as the breasts and cervix in women. In addition, scientists are studying lutein's possible role in age-related macular degeneration, cataracts, heart disease, and immune system health. Studies have also shown that lutein is associated with a reduction in lung, breast, and cervical cancer. In the vascular system, lutein is found in high-density lipoprotein (“HDL”) or “good” cholesterol and may prevent low-density lipoprotein (“LDL”) or “bad” cholesterol from oxidizing, which sets the cascade for heart disease.

Besides being a precursor of vitamin A, β-carotene is thought to be effective in helping to protect against some diseases, such as cancer, heart disease, and stroke.

Lycopene is an open-chain unsaturated carotenoid that imparts red color to tomatoes, guava, rosehip, watermelon, and pink grapefruit. Lycopene is a proven anti-oxidant that may lower the risk of certain diseases including cancer and heart disease. In the body, lycopene is deposited in the liver, lungs, prostate gland, colon, and skin. Its concentration in body tissues tends to be higher than all other carotenoids. Epidemiological studies have shown that high intake of lycopene-containing vegetables is inversely associated with the incidence of certain types of cancer. For example, habitual intake of tomato products has been found to decrease the risk of cancer of the digestive tract among Italians. In one six-year study by Harvard Medical School and Harvard School of Public Health, the diets of more than 47,000 men were studied. Of 46 fruits and vegetables evaluated, only the tomato products (which contain large quantities of lycopene) showed a measurable relationship to reduce prostate cancer risk. As consumption of tomato products increased, levels of lycopene in the blood increased, and the risk for prostate cancer decreased. Ongoing research suggests that lycopene can reduce the risk of macular degenerative disease, serum lipid oxidation, and cancers of the lung, bladder, cervix and skin. Studies are underway to investigate other potential benefits of lycopene including lycopene's potential in the fight against cancers of the digestive tract, breast, and prostate. W. Stahl & H. Sies, Lycopene: a biologically important carotenoid for humans? 336 Arch. Biochem. Biophys. 1-9 (1996); H. Gerster, The potential role of lycopene for human health, 16 J. Amer. Coll. Nutr. 109-126 (1997).

Preferred formulations and ranges of these ingredients are:

| | Ranges in Parts by Weight | | | |
|---|---|---|---|---|
| Carotenoids | Preferred | | More Preferred | |
| α-Carotene | 20-500 | μg | 20-400 | μg |
| β-Carotene | 1-20 | mg | 1-10 | mg |
| Cryptoxanthin | 5-200 | mg | 5-100 | mg |
| Lutein | 5-50 | mg | 5-20 | mg |
| Lycopene | 0.5-10 | mg | 1-10 | mg |
| Zeaxanthin | 50-1,000 | μg | 200-800 | μg |

Zinc is a trace element found in the body. It is known to occur in many important metalloenzymes. These include carbonic anhydrase, carboxypeptidases A and B, alcohol dehydrogenase, glutamic dehydrogenase, D-glyceraldehyde-3-phosphate dehydrogenase, lactic dehydrogenase, malic dehydrogenase, alkaline phosphatase, and aldolase. Impaired synthesis of nucleic acids and proteins has been observed in zinc deficiency. There is also evidence that zinc may be involved in the secretion of insulin and in the function of the hormone. Zinc is also implicated in inhibiting the PARS cycle by its involvement in repair of DNA. Y. Sheng et al., DNA Repair Enhancement by a Combined Supplement of Carotenoids, Nicotinamide and Zinc, 22 Cancer Detect. Prev. 284-292 (1998). Zinc is preferably present in the formulation in an amount in the range of about 2-200 mg and, more preferably, in the range of about 5-100 mg. Zinc may be provided in the form of inorganic salts or more bioavailable forms, such as amino acid chelates.

Niacinamide's primary mechanism of action in supporting healthy joints is through its inhibition of PARS, while NAC supports healthy joints through its potent antioxidant properties. In addition, both of these nutrients inhibit the cytokine, tumor necrosis factor-α (TNF-α), high levels of which are found in the synovial fluids and joints of certain individuals. By interrupting this cascade (FIG. 1), niacinamide and NAC help to promote healthy tissues of the joint, including cartilage and synovial fluid. Maintaining the health of these tissues in turn promotes healthy joint lubrication, flexibility, and function.

The dietary supplement of the present invention is preferably formulated as tablets. Other dosage forms known in the art, however, may also be used, such as capsules and the like. Tablets are made by combining and mixing the dry ingredients, together with any diluents, binders, lubricants, disintegrants, coloring agents, flavoring agents, and the like, and agglomerating them in an agglomeration. Similarly, wet ingredients may also be combined and mixed. Dry and wet ingredients are combined and mixed, as well. The combined, mixed, and agglomerated ingredients are then pressed into tablets according to methods well known in the art, e.g., Remington's Pharmaceutical Sciences (15^{th} ed. 1975).

The dietary supplement is preferably taken orally on a daily basis. Tablets and capsules may be taken with liquid. In a powdered formulation, the dietary supplement is preferably mixed with water, juice, or the like, and then taken orally. The preferred daily does is about 5-75 mg/kg of body weight. More preferably, the daily dose is about 10-60 mg/kg of body weight. Most preferably, the daily dose is about 20-30 mg/kg of body weight. The daily dose can be taken in one portion or can be taken as a divided dose at more than one time per day.

### EXAMPLE

The following formulations are illustrative of dietary supplements within the scope of the invention. These formulations are not to be construed as limitations on the scope of the invention, since the invention is limited only by the claims and equivalents thereof. All percentages are in percent by weight.

| Formulation A | |
|---|---|
| Niacin | 80% |
| Alpha lipoic acid | 20% |
| Formulation B | |
| Niacin | 40% |
| Niacinamide | 40% |
| Zinc chloride | 20% |
| Formulation C | |
| Niacinamide | 71.4% |
| N-acetylcysteine | 28.6% |
| Formulation D | |
| Niacinamide | 50% |
| Alpha lipoic acid | 35.7% |
| Coenzyme Q10 | 14.3% |
| Formulation E | |
| Niacin | 45.2% |
| N-acetylcysteine | 28.6% |
| Vitamin E | 7.2% |
| β-carotene | 2.9% |
| Cryptoxanthin | 14.3% |
| Lycopene | 1.8% |
| Formulation F | |
| Niacinamide | 66.7% |
| N-acetylcysteine | 25.3% |
| Lutein | 6.0% |
| Zeaxanthin | 2.0% |
| Formulation G | |
| Niacin | 42.5% |
| Niacinamide | 42.5% |
| Alpha lipoic acid | 14.3% |
| α-carotene | 0.7% |
| Formulation H | |
| Niacin | 21.2% |
| Niacinamide | 23.2% |
| N-acetylcysteine | 7.3% |
| Alpha lipoic acid | 7.3% |
| Coenzyme Q10 | 7.3% |
| Vitamin E | 2.0% |
| α-Carotene | 0.1% |
| β-Carotene | 4.0% |
| Cryptoxanthin | 14.3% |
| Lutein | 4.0% |
| Lycopene | 2.0% |
| Zeaxanthin | 0.2% |
| Zinc amino acid chelate | 7.1% |

The subject matter claimed is:

1. A dietary supplement for promoting health of joints and joint tissues comprising a mixture of an effective amount of a member selected from the group consisting of niacinamide, niacin, and mixtures thereof, and an effective amount of an antioxidant nutrient.

2. The dietary supplement of claim 1 wherein said antioxidant nutrient is a member selected from the group consisting of N-acetylcysteine, alpha lipoic acid, coenzyme Q10, vitamin E, carotenoids, zinc, and mixtures thereof.

3. The dietary supplement of claim 2 wherein said antioxidant nutrient is N-acetylcysteine.

4. The dietary supplement of claim 3 wherein said member selected from the group consisting of niacinamide, niacin, and mixtures thereof is niacinamide.

5. The dietary supplement of claim 1 further comprising a member selected from the group consisting of diluents, binders, lubricants, disintegrants, coloring agents, flavoring agents, and mixtures thereof.

6. A dietary supplement for promoting health of joints and joint tissues comprising a mixture of about 10-90% by weight of a member selected from the group consisting of niacinamide, niacin, and mixtures thereof and about 10-90% by weight of an antioxidant nutrient.

7. The dietary supplement of claim 6 wherein said antioxidant nutrient is a member selected from the group consisting of N-acetylcysteine, alpha lipoic acid, coenzyme Q10, vitamin E, carotenoids, zinc, and mixtures thereof.

8. The dietary supplement of claim 7 wherein said antioxidant nutrient is N-acetylcysteine.

9. The dietary supplement of claim 8 wherein said member selected from the group consisting of niacinamide, niacin, and mixtures thereof is niacinamide.

10. The dietary supplement of claim 6 further comprising an effective amount of a member selected from the group consisting of diluents, binders, lubricants, disintegrants, coloring agents, flavoring agents, and mixtures thereof.

11. The dietary supplement of claim 6 comprising about 30-85% by weight of said member selected from the group consisting of niacinamide, niacin, and mixtures thereof and about 15-70% by weight of said antioxidant nutrient.

12. The dietary supplement of claim 11 comprising about 50-80% by weight of said member selected from the group consisting of niacinamide, niacin, and mixtures thereof and about 20-50% by weight of said antioxidant nutrient.

13. A method for promoting healthy joints and joint tissues comprising orally administering an effect amount of a dietary supplement comprising a mixture of an effective amount of a member selected from the group consisting of niacinamide, niacin, and mixtures thereof and an effective amount of an antioxidant ingredient.

14. The method of claim 13 wherein said effective amount of said dietary supplement comprises about 5-75 mg/kg/day.

15. The method of claim 13 wherein said dietary supplement comprises about 10-90% by weight of said member selected from the group consisting of niacinamide, niacin, and mixtures thereof and about 10-90% by weight of said antioxidant ingredient.

16. The method of claim 13 wherein said member selected from the group consisting of niacinamide, niacin, and mixtures thereof is niacinamide.

17. The method of claim 13 wherein said antioxidant nutrient is a member selected from the group consisting of N-acetylcysteine, alpha lipoic acid, coenzyme Q10, vitamin E, carotenoids, zinc, and mixtures thereof.

18. The method of claim 17 wherein said antioxidant nutrient is N-acetylcysteine.
